(19) United States
(12) Patent Application Publication
(10) Pub. No.: US 2026/0069305 A1
(43) Pub. Date: Mar. 12, 2026

(54) NEEDLE UNIT FOR PROCEDURE USING MAGNETIC FORCE

(71) Applicant: THE CATHOLIC UNIVERSITY OF KOREA INDUSTRY-ACADEMIC COOPERATION FOUNDATION, Seoul (KR)

(72) Inventors: Jae Min KIM, Seoul (KR); In Jong KIM, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 19/276,233

(22) Filed: Jul. 22, 2025

(30) Foreign Application Priority Data
Sep. 9, 2024 (KR) 10-2024-0122728

(51) Int. Cl. A61B 17/32; A61B 17/00

(52) U.S. Cl. CPC A61B 17/320036; A61B 2017/00477; A61B 2017/00876

(57) ABSTRACT

Disclosed is a needle unit for a procedure using a magnetic force, the needle unit including a first needle having a first passage formed therein through which a cutting thread for cutting a biological tissue of an affected area is moved and having a first needle tip formed at a tip thereof, a second needle having a second passage formed therein which communicates with the first passage and through which the cutting thread is moved and having a second needle tip formed at a tip thereof and connected to the first needle tip, and a magnetic member that connects the first needle tip and the second needle tip using a magnetic force so that the first passage and the second passage communicate with each other, and forms a loop in which the cutting thread is moved.

### CROSS-REFERENCE TO RELATED APPLICATIONS

A claim for priority under 35 U.S.C. § 119 is made to Korean Patent Application No. 10-2024-0122728 filed on Sep. 9, 2024 in the Korean Intellectual Property Office, the entire contents of which are hereby incorporated by reference.

### BACKGROUND

Embodiments of the present disclosure described herein relate to a needle unit for a procedure using a magnetic force to mount a cutting thread for cutting a biological tissue of an affected area onto a desired biological tissue while minimizing damage to surrounding tissues.

In recent years, the number of people working on computers or using mobile phones using wrists thereof is increasing, and thus the number of patients having carpal tunnel syndromes and trigger finger syndromes is increasing.

In order to treat affected areas of these patients, in some cases, a procedure of cutting a biological tissue of the affected area using a cutting thread is required.

Meanwhile, in order to cut the biological tissue of the affected area, a process of mounting the cutting thread on the biological tissue is required.

In the related art, in order to mount the cutting thread in the biological tissue of the affected area, first, a first needle is inserted through the affected area, the cutting thread passes through the first needle and is withdrawn from the first needle by a certain length, and the first needle is removed from the affected area. In this case, a portion of the cutting thread withdrawn from the first needle is exposed to the outside of the affected area, and the other portion of the cutting thread remains in the biological tissue.

Next, a second needle is inserted through the affected area, the cutting thread withdrawn from the first needle passes through the second needle and is withdrawn from the second needle by a certain length, and then the second needle is removed from the affected area. In this case, a portion of the cutting thread withdrawn from the second needle is exposed to the outside of the affected area, and the other portion of the cutting thread remains in the biological tissue.

Accordingly, the cutting thread forms a single loop in the affected area and is mounted on the biological tissue of the affected area, and opposite ends of the cutting thread are exposed from the affected area.

Further, an operator may remove the biological tissue of the affected area by holding and reciprocating the opposite ends of the cutting thread exposed from the affected area.

However, in a process of cutting the biological tissue using the cutting thread according to the related art, the cutting thread is exposed from a skin and is then inserted into the skin again, and thus the skin from which the cutting thread is withdrawn and a surrounding soft tissue on which the cutting thread is mounted are damaged.

### SUMMARY

Embodiments of the present disclosure provide a needle unit for a procedure using a magnetic force capable of minimizing damage to surrounding tissues, easily performing a procedure, shortening a procedure time, and improving patient satisfaction.

The aspects of the present disclosure are not limited to the aspects described above, and those skilled in the art will clearly understand other aspects not described from the following description.

According to an embodiment, a needle unit for a procedure using a magnetic force includes a first needle having a first passage formed therein through which a cutting thread for cutting a biological tissue of an affected area is moved and having a first needle tip formed at a tip thereof, a second needle having a second passage formed therein which communicates with the first passage and through which the cutting thread is moved and having a second needle tip formed at a tip thereof and connected to the first needle tip, and a magnetic member that is provided in a connection area between the first needle tip and the second needle tip, connects the first needle tip and the second needle tip using a magnetic force so that the first passage and the second passage communicate with each other, and forms a loop in which the cutting thread is moved.

Here, as an embodiment, the magnetic member may include one or more magnets that are provided on at least one of a tip of the first needle and a tip of the second needle and generate a magnetic force.

Further, as another embodiment, the first needle and the second needle may be made of a metal material so that the first needle and the second needle are coupled to each other by a magnetic force, and the magnetic member may include a magnet that is provided outside the connection area between the first needle tip and the second needle tip and connects the first needle tip and the second needle tip using a magnetic force.

The first needle and the second needle may have the same diameter or different diameters.

A tip of the first needle and a tip of the second needle may have a partial arc shape and be bent to be curved.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a diagram of a needle unit for a procedure according to an embodiment of the present disclosure; and

FIGS. 2 to 6 are views illustrating a process of mounting a cutting thread on a biological tissue using a needle unit for a procedure according to the embodiment of the present disclosure.

### DETAILED DESCRIPTION

Advantages and features of the present disclosure and a method of achieving the advantages and the features will become apparent with reference to embodiments described below in detail together with the accompanying drawings. However, the present disclosure is not limited to embodiments disclosed below but may be implemented in various different forms, and the embodiments are provided only to make the disclosure of the present disclosure complete and to completely inform the scope of the present disclosure to those skilled in the art to which the present disclosure pertains.

Terms used in the specification are intended to describe embodiments and are not intended to limit the present disclosure. In the specification, a singular form also includes a plural form unless specifically mentioned in a phrase. The terms “comprises” and/or “comprising” used in the specification do not exclude the presence or addition of one or more other components other than the mentioned components. Throughout the specification, the same reference numerals refer to the same components, and the term “and/or” includes each and all combinations of one or more of components mentioned.

Unless otherwise defined, all terms (including technical and scientific terms) used in the specification may be used as meanings commonly understood by those skilled in the art to which the present disclosure pertains. Further, terms defined in a commonly used dictionary are not interpreted ideally or excessively unless explicitly and specifically defined.

Hereinafter, the present disclosure will be described in detail with reference to the accompanying drawings.

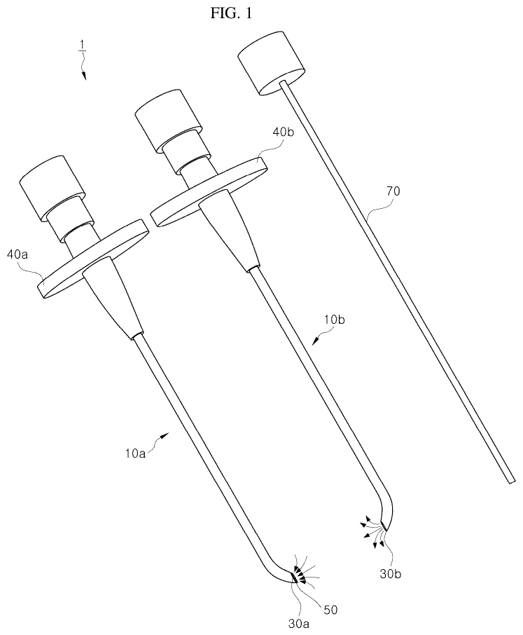

FIG. 1 illustrates a needle unit for a procedure using a magnetic force according to an embodiment of the present disclosure.

As illustrated in the drawings, a needle unit 1 for a procedure using a magnetic force according to the embodiment of the present disclosure includes a first needle 10a, a second needle 10b, and a magnetic member 50.

The first needle 10a has a predetermined length and a predetermined diameter and has a hollow cannula shape.

A first passage 20a is formed through an inside of the first needle 10a in a longitudinal direction of the first needle 10a.

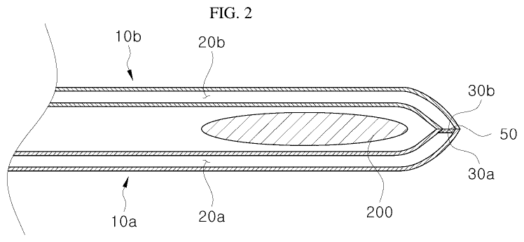

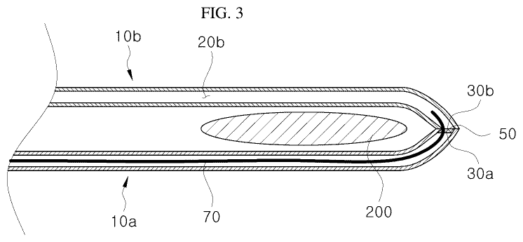

A cutting thread 100 (see FIG. 3) for cutting a biological tissue 200 (see FIG. 2) of an affected area is movably provided in the first passage 20a.

Meanwhile, a first needle tip 30a for needling the affected area is formed at a tip of the first needle 10a.

The tip of the first needle 10a has a partial arc shape having a certain curvature and is formed to be curved.

A handle 40a for gripping the first needle 10a is provided at the other end opposite to the tip of the first needle 10a.

An auxiliary passage (not illustrated) through which the cutting thread 100 for cutting the biological tissue 200 may be introduced into the first passage 20a of the first needle 10a may be formed in the handle 40a.

Accordingly, the first needle 10a has a tuohy needle shape.

Here, the handle 40a of the first needle 10a is not limited to the shape illustrated in the drawing and may have various shapes.

The second needle 10b has the same shape as the first needle 10a.

That is, the second needle 10b has a predetermined length and a predetermined diameter and has a hollow cannula shape.

A second passage 20b is formed through an inside of the second needle 10b in a longitudinal direction of the second needle 10b.

The second passage 20b communicates with the first passage 20a of the first needle 10a and is provided such that the cutting thread 100 for cutting the biological tissue 200 of the affected area is movable.

Meanwhile, a second needle tip 30b that needles the affected area and is connected to the first needle tip 30a of the first needle 10a is formed at a tip of the second needle 10b.

The tip of the second needle 10b has a partial arc shape having a certain curvature and is formed to be curved.

A handle 40b for gripping the second needle 10b is provided at the other end opposite to the tip of the second needle 10b.

An auxiliary passage (not illustrated) through which the cutting thread 100 for cutting the biological tissue 200 passing through the second passage 20b of the second needle 10b is withdrawn may be formed in the handle 40b.

Accordingly, the second needle 10b also has a tuohy needle shape.

Here, the handle 40b of the second needle 10b is not limited to the shape illustrated in the drawing and may have various shapes.

Meanwhile, the second needle 10b may have the same diameter as or a different diameter from that of the first needle 10a. As an example, the second needle 10b may have a diameter smaller than that of the first needle 10a and thus may be needled in the affected area while minimizing damage to surrounding tissues.

The magnetic member 50 is provided in a connection area between the first needle tip 30a of the first needle 10a and the second needle tip 30b of the second needle 10b and generates a magnetic force so that the first needle tip 30a and the second needle tip 30b are connected to each other.

The magnetic member 50 includes a magnet that is provided on the first needle tip 30a at the tip of the first needle 10a and generates a magnetic force. Here, the magnet may be provided at the tip of the first needle 10a adjacent to the first needle tip 30a instead of the first needle tip 30a of the first needle 10a.

Accordingly, the second needle 10b made of a metal material coupled by a magnetic force is connected to the first needle 10a by a magnetic force generated by the magnetic member 50 provided on the first needle 10a. In more detail, the first needle tip 30a of the first needle 10a and the second needle tip 30b of the second needle 10b, which are inserted into a skin of the affected area and are arranged to face each other, are connected to each other by a magnetic force generated by the magnetic member 50 provided in the first needle 10a, the first passage 20a of the first needle 10a and the second passage 20b of the second needle 10b communicate with each other, and thus a loop in which the cutting thread 100 is moved is formed.

Here, as another embodiment, although not illustrated, the magnetic member may include magnets having different polarities at the tip of the first needle and the tip of the second needle, the first needle tip of the first needle and the second needle tip of the second needle, which are arranged into the skin of the affected area and face each other, may be connected to each other by a magnetic force generated by the magnet, and thus the first passage of the first needle and the second passage of the second needle may communicate with each other.

Further, as still another embodiment, although not illustrated, the first needle and the second needle may be made of a metal material so that the first needle and the second needle are coupled to each other by a magnetic force, the magnetic member includes a magnet provided outside the skin of the affected area to which the first needle tip and the second needle tip are connected, the first needle tip of the first needle and the second needle tip of the second needle, which are inserted into the skin of the affected area, may be connected to each other by a magnetic force outside the skin of the affected area, and accordingly, the first passage of the first needle and the second passage of the second needle may communicate with each other.

According to this configuration, to help understand the disclosure of the needle unit 1 for a procedure using a magnetic force according to the embodiment of the present disclosure, as an example, a process of mounting the cutting thread 100 for cutting the biological tissue 200 of the affected area on the biological tissue 200 will be described briefly using the needle unit 1 for a procedure using a magnetic force according to the embodiment of the present disclosure.

First, as illustrated in FIG. 2, the first needle 10a is inserted into a lower portion of the biological tissue 200 of the affected area.

Next, the second needle 10b is inserted into an upper portion of the biological tissue 200 of the affected area so as not to interfere with the first needle 10a.

In this case, the second needle 10b is inserted so that the second needle tip 30b of the second needle 10b faces the first needle tip 30a of the first needle 10a.

When the second needle tip 30b of the second needle 10b is located to face the first needle tip 30a of the first needle 10a, the first needle tip 30a and the second needle tip 30b come into contact with each other by a magnetic force of the magnetic member 50, the first passage 20a of the first needle 10a and the second passage 20b of the second needle 10b communicate with each other, and thus a loop having a “U” shape through which the cutting thread 100 is moved is formed.

Here, in a process of connecting the first needle 10a and the second needle 10b, when a cartilage tissue is present between the first needle tip 30a of the first needle 10a and the second needle tip 30b of the second needle 10b, as illustrated in FIG. 3, the cartilage tissue may be drilled with a styllet 70 introduced into the first needle 10a, the first needle 10a and the second needle 10b may be arranged to face each other, and the first needle 10a and the second needle 10b may be magnetically coupled to each other by a magnetic force of the magnetic member 50.

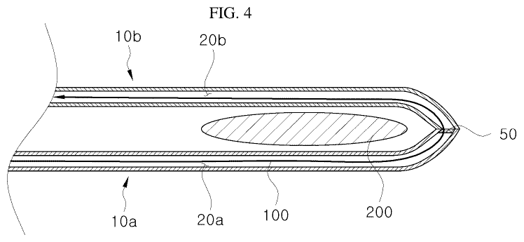

Next, the cutting thread 100 is bound to an inner needle (not illustrated), and as illustrated in FIG. 4, the cutting thread 100 bound to the inner needle is introduced into the second needle 10b through the second needle tip 30b via the first needle tip 30a along the first passage 20a of the first needle 10a, and then is withdrawn to the outside of the second needle 10b via the second passage 20b of the second needle 10b.

Accordingly, in the needle unit 1 for a procedure according to the present disclosure, the cutting thread 100 is not exposed to the outside and is inserted into the first passage 20a of the first needle 10a and the second passage 20b of the second needle 10b, thereby preventing the risk of bacterial infection due to external exposure of the cutting thread 100.

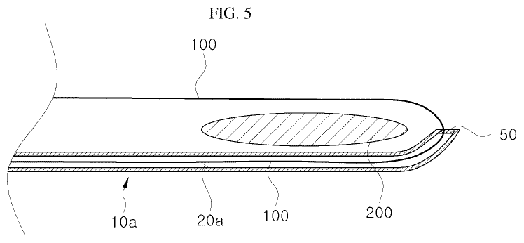

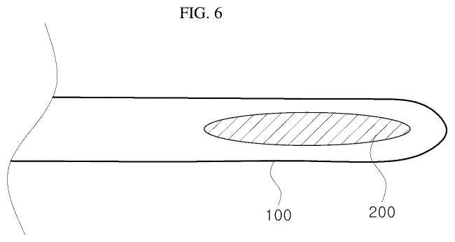

Further, the second needle 10b inserted into the affected area and magnetically coupled to the first needle 10a in a state in which the first needle 10a is fixed so that only the cutting thread 100 remains in the affected area is withdrawn from the affected area as illustrated in FIG. 5, and then the first needle 10a inserted into the affected area is withdrawn from the affected area as illustrated in FIG. 6.

Accordingly, the cutting thread 100 may form a “U” shaped loop and be mounted on the biological tissue 200 of the affected area, and thus, an operator may cut the biological tissue 200 of the affected area using the cutting thread 100,

In this way, according to the present disclosure, the first needle tip of the first needle and the second needle tip of the second needle may be connected by a magnetic force to form a loop in which the cutting thread is moved, thereby minimizing damage to the surrounding tissues of the affected area, easily performing a procedure, shortening a procedure time, and improving patient satisfaction.

Further, the needle unit for a procedure using a magnetic force according to the present disclosure is very effective to cut the biological tissue of the affected area in a procedure of the carpal tunnel syndrome and the trigger finger syndrome.

Meanwhile, in the above-described embodiment, it is illustrated that the first needle and the second needle are connected to each other using a magnetic force through the magnetic member to form a loop in which the cutting thread is moved. However, as another example, when the first needle and the second needle have different diameters, the first needle and the second needle are connected to each other while checking the first needle and the second needle with ultrasonic waves without using the magnetic member to form a loop in which the cutting thread is moved.

Hereinabove, the embodiments of the present disclosure have been described with reference to the accompanying drawings. However, those skilled in the art to which the present disclosure pertains may understand that the present disclosure may be implemented in other specific forms without changing the technical spirit or essential features thereof. Therefore, it should be understood that the embodiments described above are illustrative but not limiting in all aspects.

What is claimed is:

1. A needle unit for a procedure using a magnetic force, the needle unit comprising:
a first needle having a first passage formed therein through which a cutting thread for cutting a biological tissue of an affected area is moved and having a first needle tip formed at a tip thereof;
a second needle having a second passage formed therein which communicates with the first passage and through which the cutting thread is moved and having a second needle tip formed at a tip thereof and connected to the first needle tip; and
a magnetic member provided in a connection area between the first needle tip and the second needle tip, configured to connect the first needle tip and the second needle tip using a magnetic force so that the first passage and the second passage communicate with each other, and configured to form a loop in which the cutting thread is moved.

2. The needle unit of claim 1, wherein the magnetic member includes one or more magnets provided on at least one of a tip of the first needle and a tip of the second needle and configured to generate a magnetic force.

3. The needle unit of claim 1, wherein the first needle and the second needle are made of a metal material so that the first needle and the second needle are coupled to each other by a magnetic force, and the magnetic member includes a magnet provided outside the connection area between the first needle tip and the second needle tip and configured to connect the first needle tip and the second needle tip using a magnetic force.

4. The needle unit of claim 1, wherein the first needle and the second needle have the same diameter or different diameters.

5. The needle unit of claim 1, wherein a tip of the first needle and a tip of the second needle have a partial arc shape and are bent to be curved.
